(19) United States
(12) Patent Application Publication
(10) Pub. No.: US 2005/0283226 A1
(43) Pub. Date: Dec. 22, 2005

(54) Medical devices

(75) Inventor: Patrick A. Haverkost, Brooklyn Center, MN (US)

Correspondence Address: FISH & RICHARDSON PC, P.O. BOX 1022, MINNEAPOLIS, MN, 55440-1022, US

(73) Assignee: Scimed Life Systems, Inc., Maple Grove, MN (US)

(21) Appl. No.: 10/872,164

(22) Filed: Jun. 18, 2004

(51) Int. Cl. A61F 2/06

(52) U.S. Cl. 623/1.15; 623/1.34

(57) ABSTRACT

Medical devices, particularly stents, including a polymer body with radiopaque material are disclosed.

### TECHNICAL FIELD

This invention relates to medical devices, such as, for example, endoprostheses.

### BACKGROUND

The body includes various passageways such as arteries, other blood vessels, and other body lumens. For various treatments and diagnostic techniques, it is often desirable to deliver a medical device into these lumens. For example, these passageways sometimes become occluded or weakened. The passageways can be occluded by e.g. a tumor, restricted by plaque, or weakened by an aneurysm. When this occurs, the passageway can be reopened or reinforced, or even replaced, with a medical endoprosthesis.

An endoprosthesis is typically a tubular member that is placed in a lumen in the body. Examples of endoprostheses include stents and covered stents, sometimes called “stent-grafts”. An endoprosthesis can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen. The expansion mechanism may include forcing the endoprosthesis to expand radially. For example, the expansion mechanism can include the catheter carrying a balloon, which carries the endoprosthesis. The balloon can be inflated to deform and to fix the expanded endoprosthesis at a predetermined position in contact with the lumen wall. The balloon can then be deflated, and the catheter removed.

In another delivery technique, the endoprosthesis is self-expanding. For example, the endoprosthesis can be formed of an elastic material that can be reversibly compacted and expanded. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force. Another self-expansion technique uses shape memory metals which can “remember” a particular geometric configuration, e.g. an expanded condition, upon exposure to a trigger, such as an increase in temperature.

### SUMMARY

In one aspect, the invention features a medical stent with a stent body including a generally tubular member, the generally tubular member including a wall that defines at least one void, and a radiopaque material bonded to the stent body by a polymer.

In another aspect, the invention features a medical stent with a stent body including a generally tubular member, the generally tubular member having a wall that defines at least one void. The medical stent also includes a radiopaque material that is bonded to the stent body by a polymer. The polymer spans the void, and the radiopaque material is suspended within the void.

In another aspect, the invention features a medical stent with a stent body that defines a generally tubular member and that includes a pattern of voids defined through a tubular stent wall. The geometry and/or location of the voids are selected to facilitate expansion and/or contraction of the stent. The medical stent also includes a radiopaque marker suspended within one of the voids. The radiopaque marker renders the medical stent radiopaque independently of the stent body.

In another aspect, the invention features a method of making a stent, the method including combining a radiopaque material with a first polymer, and attaching the first polymer to an end of a stent body defining a generally tubular member. The generally tubular member has a wall that defines at least one void. The first polymer spans the void, and the radiopaque material is suspended within the void.

In other aspects, the invention features a medical device including a void, and a polymer that e.g. spans the void, and a radiopaque material suspended within the void. The medical device may include, for example, a plurality of voids. Examples include mesh-forms, such as filters, embolic protection devices, and valves.

Embodiments can include one or more of the following features.

The generally tubular member can include a pattern of voids defined through a tubular stent wall, and radiopaque material can be suspended within a plurality of the voids. The radiopaque material (e.g., the radiopaque marker) can be proximate an end or both ends of the stent body. The medical stent can include a plurality of radiopaque markers, and each radiopaque marker can be suspended within a void and located proximate an end of the stent body. The polymer can include a continuous element that extends over about 50 percent or more of the circumference of the stent body. The polymer can be in the shape of a ring. The ring can have a thickness of about 125 percent of the thickness of the stent body or less, and/or a width of about 25 percent of the length of the stent body or less. The ring can include at least two layers of polymeric material. The polymer can be shaped to complement an edge of the stent body. The polymer can be a fluoropolymer (e.g., expanded-polytetrafluoroethylene). The polymer can encapsulate the radiopaque material. The radiopaque material can be dispersed in the polymer. The radiopaque material can include a body of radiopaque metal. The body of radiopaque metal (e.g., the radiopaque marker) can have a thickness of about 110 percent of the thickness of the stent body or less, and about 75 percent of the thickness of the stent body or more. The body of radiopaque metal can have a thickness of from about 0.001 inch to about 0.01 inch (e.g., from about 0.005 inch to about 0.008 inch). The radiopaque material can be a metal (e.g., tungsten, tantalum, platinum, palladium, lead, gold, titanium, silver), a metal alloy, a metal oxide, bismuth subcarbonate, or barium sulfate. The radiopaque material can have a density of about ten grams per cubic centimeter or greater. The medical stent can further include a therapeutic agent. The generally tubular member and/or the polymer can include the therapeutic agent.

The method can include providing a first strip of the first polymer, positioning a plurality of radiopaque markers on the first strip of the first polymer, and attaching the first strip to the stent body. The method can include positioning the radiopaque markers on the first strip at locations that correspond to voids defined by the stent body. The attachment of the first strip to the stent body can include assembling the first strip in contact with the stent body and bonding the first strip to the stent body. The first strip can be attached to the stent body by an adhesive, by melting, and/or by sintering or partially sintering the first strip. The method can include attaching the first strip to a second strip. The second strip can include a second polymer. The method can include attaching the first strip to the second strip with an adhesive. The method can include melt-bonding the first strip to the second strip. The method can include sintering or partially sintering the first strip to the second strip. The first polymer and the second polymer can be different polymers. The method can further include applying the second strip to at least one radiopaque marker to encapsulate the radiopaque marker. Combining a radiopaque material with a first polymer can include dispersing the radiopaque material in the first polymer. Combining a radiopaque material with a first polymer can include attaching (e.g., adhering) at least one radiopaque marker to the first polymer. Adhering a radiopaque marker to the first polymer can include spraying the radiopaque marker with a dispersion and/or dipping the radiopaque marker in a dispersion, and placing the radiopaque marker on the first polymer. The dispersion can include tetrafluoroethylene or fluorinated ethylene propylene (FEP). Attaching at least one radiopaque marker to the first polymer can include heating the radiopaque marker and the first polymer. The method can include positioning at least one radiopaque marker in a void that is defined by the stent body. The first polymer can include a fluoropolymer (e.g., expanded-polytetrafluoroethylene). Attaching the first polymer to an end of a stent body can include sintering or partially sintering the first polymer to the end of the stent body. The method can further include contouring an edge of the first polymer.

Embodiments can include one or more of the following advantages.

In some embodiments, the location of an endoprosthesis with a polymer body that includes radiopaque material can be readily ascertained (e.g., by using x-ray fluoroscopy). In certain embodiments (e.g., embodiments in which both ends of an endoprosthesis include polymer rings with T-shaped radiopaque markers), both the location and the orientation of an endoprosthesis can be readily ascertained.

An endoprosthesis with a polymer body that includes radiopaque material can have a low profile. In some embodiments, a polymer body that includes radiopaque markers can be attached to an endoprosthesis without substantially increasing the profile (e.g., the deployment diameter) of the endoprosthesis. In certain embodiments, an endoprosthesis with a polymer body that includes radiopaque material (e.g., radiopaque markers) can provide more space for the radiopaque material than an endoprosthesis that lacks such a polymer body. As a result, the endoprosthesis with the polymer body may be adapted to incorporate more radiopaque material than the endoprosthesis that does not include the polymer body.

Radiopaque material that is incorporated into a polymer body in an endoprosthesis may be less likely to detach from the endoprosthesis than radiopaque material that is not incorporated into a polymer body. Thus, the endoprosthesis with the polymer body may have a relatively low likelihood of inflicting harm during use (e.g., by eliciting emboli formation).

An endoprosthesis with a polymer body incorporating radiopaque material may not require an extra structure or structures within its endoprosthesis body to hold the radiopaque material.

An endoprosthesis with a polymer body (made of, e.g., expanded polytetrafluoroethylene) at one or both of its ends can be less likely to result in stent end effects (harm to the body lumen, such as injury to body tissue, resulting from contact with one or both ends of the stent) than an endoprosthesis that does not have a polymer body at one or both of its ends. The polymer body can cover, e.g., pointed stent ends, making them less likely to harm surrounding tissue. In some embodiments, an endoprosthesis that includes a polymer body can withstand fatigue better than an endoprosthesis without such a polymer body.

An endoprosthesis with a polymer body at one or both of its ends that includes radiopaque material can be quickly and/or inexpensively produced, relative to an endoprosthesis that includes radiopaque material but lacks such a polymer body. In some embodiments, the manufacturing throughput of an endoprosthesis with a polymer body at one or both of its ends that includes radiopaque material can be relatively high.

In embodiments, a polymer body that includes radiopaque material can be relatively easy to assemble. In some embodiments, an endoprosthesis that includes the polymer body can be easier to assemble than, for example, an endoprosthesis with radiopaque markers that require attachment at several locations on and/or within the endoprosthesis body.

Still further aspects, features, and advantages follow.

### DESCRIPTION OF DRAWINGS

FIG. 1A is a perspective view of a stent.

FIG 1B is a side view of the stent of FIG. 1A.

FIG 1C is an enlarged view of region 1C in FIG 1B.

FIG 1D is a cross-sectional view of region 1C, taken along line 1D-1D.

FIGS. 2A-2H are schematic views of the assembly of a stent.

FIGS. 3A-3C are schematic views of the assembly of a stent.

FIGS. 4A-4C illustrate delivery of a self-expanding stent.

FIGS. 5A-5C illustrate delivery of a balloon-expandable stent.

FIGS. 6A and 6B illustrate a method of forming a stent.

### DETAILED DESCRIPTION

Structure

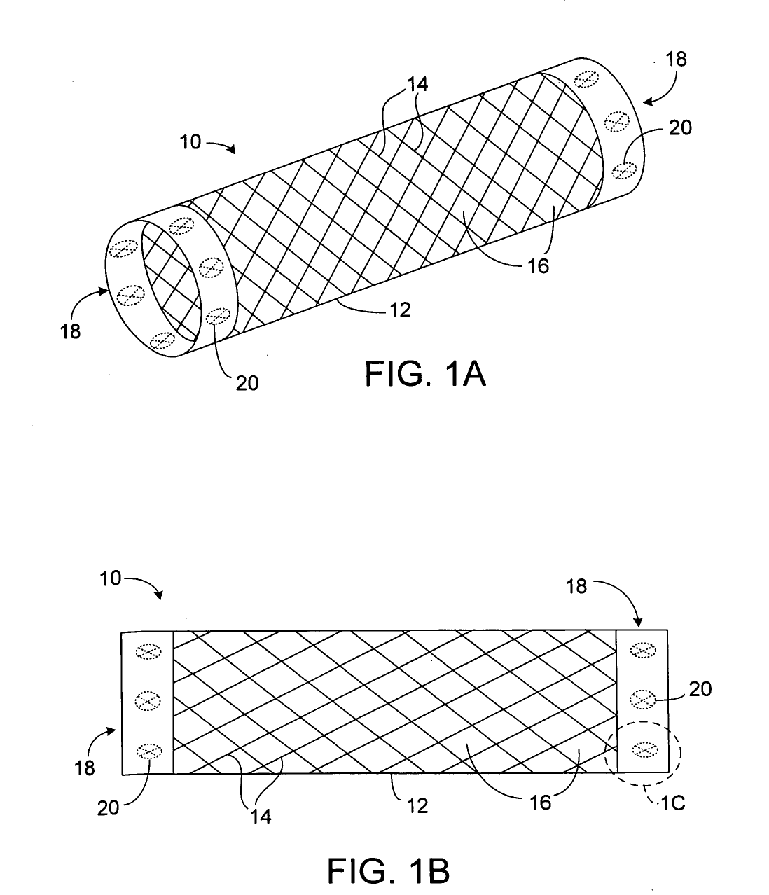

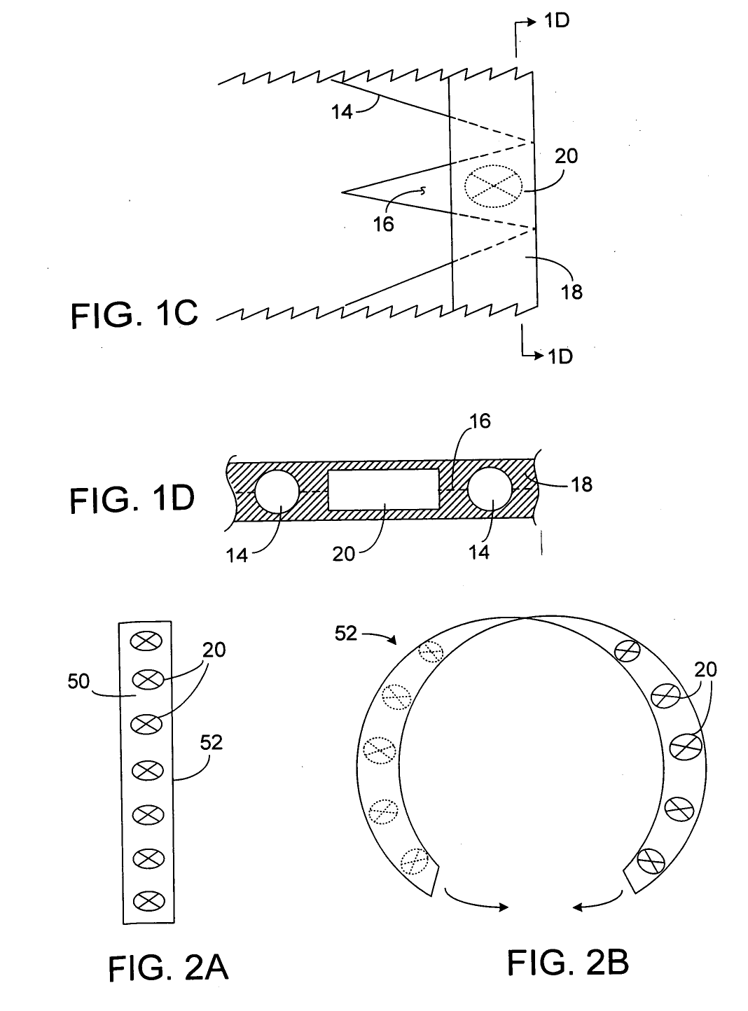

Referring to FIGS. 1A and 1B, a stent 10 includes a generally tubular stent body 12 formed of strand materials 14. Strand materials 14 define a pattern of voids 16 in the wall of stent body 12. Voids 16 facilitate the expansion and contraction of stent 10, and enhance the flexibility of stent 10. At each of its ends, stent 10 includes a polymer body 18 in the shape of a ring that is attached to stent body 12. Radiopaque markers 20, in the form of solid metal slugs, are embedded in polymer body 18. A plurality of markers are spread circumferentially around the stent ends.

Referring as well to FIGS. 1C and 1D, markers 20 are positioned within voids 16 such that markers 20 do not overlap with, or contact, strand materials 14. Furthermore, markers 20 have approximately the same thickness as strand materials 14. As a result, a relatively thick body of radiopaque material can be provided without substantially increasing the thickness profile of stent 10.

The markers 20 include one or more radiopaque materials to enhance the visibility of stent 10 under x-ray fluoroscopy. A radiopaque material can be, for example, a metal (e.g., tungsten, tantalum, platinum, palladium, lead, gold, titanium, silver); a metal alloy (e.g., stainless steel, an alloy of tungsten, an alloy of tantalum, an alloy of platinum, an alloy of palladium, an alloy of lead, an alloy of gold, an alloy of titanium, an alloy of silver); a metal oxide (e.g., titanium dioxide, zirconium oxide, aluminum oxide); bismuth subcarbonate; or barium sulfate. In some embodiments, a radiopaque material can be a metal with a density of about ten grams per cubic centimeter or greater (e.g., about 25 grams per cubic centimeter or greater, about 50 grams per cubic centimeter or greater). The radiopaque material is provided as a solid metal slug and/or a radiopaque powder distributed in the polymer body. Suitable radiopaque materials are discussed in Heath, U.S. Pat. No. 5,725,570, the entire contents of which are hereby incorporated by reference.

The thickness and width of the markers provide a desirable radiographic image. In embodiments, the thickness of one or more of the markers is comparable to the thickness of the stent body. For example, the thickness of the marker is about ±25 percent, about ± ten percent, about ± five percent, or less than the thickness of the stent body. In embodiments, the thickness is from about 0.001 inch to about 0.01 inch (e.g., from about 0.005 inch to about 0.008 inch). In embodiments, the width of the markers is such that the markers can be positioned within the voids of the stent body without contacting or overlapping the stent body when the stent is in an expanded, implanted condition. In embodiments, the markers are sized to be positioned within the voids without contacting or overlapping the stent body when the stent is in a collapsed, delivery condition and an expanded, implanted condition. In particular embodiments, the width of the markers is 90 percent or less, e.g., 50 percent or less or ten percent or less than the width of the voids in the expanded and/or contracted condition. In particular embodiments, the maximum width of the markers is about two millimeters or less, e.g., one millimeter or less or one millimeter to 0.1 millimeter. Preferably, markers located at the ends of the stent do not extend substantially beyond the periphery of the stent body, so that the length of the stent is not increased. In embodiments, the markers extend less than about two millimeters beyond the length of the stent body (e.g., less than about 1.5 millimeters, less than about one millimeter, less than about 0.5 millimeter). In embodiments, the markers are discrete elements (e.g., metal slugs) that provide sufficient radiopacity independently of the stent body (without requiring the presence of the stent body) to provide a desirable radiopaque image.

The location, shape, and number of markers provide a particular radiographic image. To indicate one or both ends of the stent, markers are provided at the ends of the stent. In embodiments, markers are provided along the body of the stent at predetermined distances from the end of the stent. A single marker or multiple markers can be provided along the stent axis and/or circumferentially about the axis. A pattern of markers can provide an indication of stent orientation about the axis. The markers can be shaped to indicate orientation, e.g. cylindrical, disk-shaped or T-shaped markers can be provided. In some embodiments, the markers can be in the form of radiopaque wires (e.g., individual radiopaque wires or bundles of radiopaque wires). In certain embodiments, the radiopaque wire markers can have a diameter of from about 0.001 inch to about 0.015 inch (e.g., about 0.01 inch), and/or a length of from about 0.5 millimeter to about two millimeters, and/or an aspect ratio (the ratio of the length of the radiopaque wire markers to the diameter of the radiopaque wire markers) of from about 1/1 to about 20/1. In certain embodiments, the radiopaque wire markers can have rounded or tumbled edges. In embodiments, one or more of the radiopaque wire markers can be in the form of a coil. Markers of different shapes can be used on the same stent.

The polymer body is biocompatible, compatible with the radiopaque material incorporated in the polymer body, of sufficient strength to retain the markers, and of sufficient flexibility to accommodate stent expansion and flexing during delivery or after implantation. The polymer body is formed of one or more layers of a polymer such as a fluoropolymer (e.g., expanded-polytetrafluoroethylene), Corethane®, a polyisobutylene-polystyrene block copolymer such as SIBS (see, e.g., U.S. Pat. No. 6,545,097), fluorinated ethylene propylene (FEP), tetrafluoroethylene (TFE), and silicone (e.g., in embodiments of stent 10 that are used for non-vascular applications). The thickness of the polymer body is sufficient to securely retain and bond the marker to the stent body. The polymer body bonds to portions of the stent body adjacent a void in which a marker is positioned. In embodiments, the polymer overlaps the adjacent regions. The thickness of the overlap region is selected to reduce the overall thickness profile of the stent. In embodiments, the thickness of the overlap region on an exterior wall surface of the stent is 25 percent or less, e.g., ten percent or one percent or less than the thickness of the stent wall. In particular embodiments, the thickness of the overlap region is about 200 microns or less. In embodiments, the thickness of the portions of the polymer body overlapping the marker similarly does not greatly increase the thickness profile of the stent. The polymer body extends in particular embodiments into the void between the marker and the stent body to prevent direct contact between the marker and the stent body. The polymer body can include a drug, e.g. an antiproliferative, that elutes from the polymer body into adjacent tissue to, e.g., inhibit restenosis.

In embodiments, the polymer body can extend over from about ten percent to about 100 percent of the circumference of stent body 12, e.g. more than 50 percent. The width of the polymer body along the stent axis extends over about one percent to 100 percent of the length of the stent. In particular embodiments, the width of the polymer body is about ten millimeters or less, e.g., about two millimeters.

The polymer body can be formed and bonded to the stent by solvent casting, or dipping a suitable polymer directly onto the stent. Alternatively, a preformed polymer body can be bonded to the stent. In particular embodiments, the polymer body is formed from one or more preformed polymer strips. In particular embodiments, the markers are sandwiched between the strips, which are bonded together by an adhesive or co-melted, and/or which are sintered or partially sintered together.

In certain embodiments, a stent body can be formed of strands. The strands can be, e.g., woven, knitted, or crocheted. In embodiments, a stent body can be in the form of a sheet-form body with apertures (formed by, e.g., cutting or etching). The stent body can be defined by a metal or a polymer. The stent can be self-expanding or balloon expandable. Stents are further described in Heath, incorporated sulpra, and Wang, U.S. Pat. No. 6,379,379, the entire contents of which are hereby incorporated by reference.

Manufacture

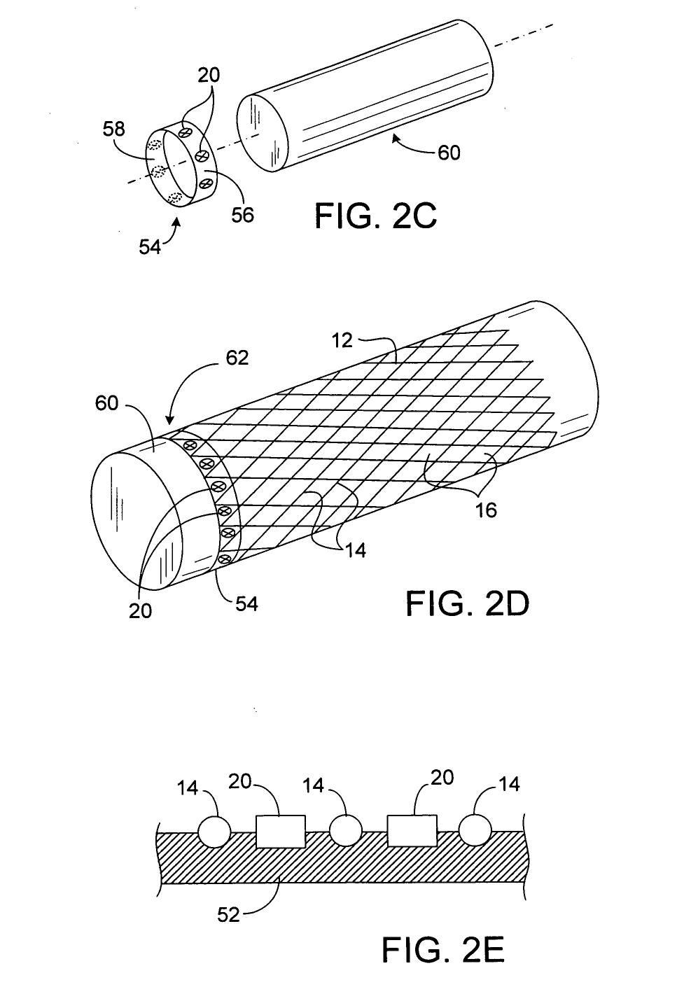

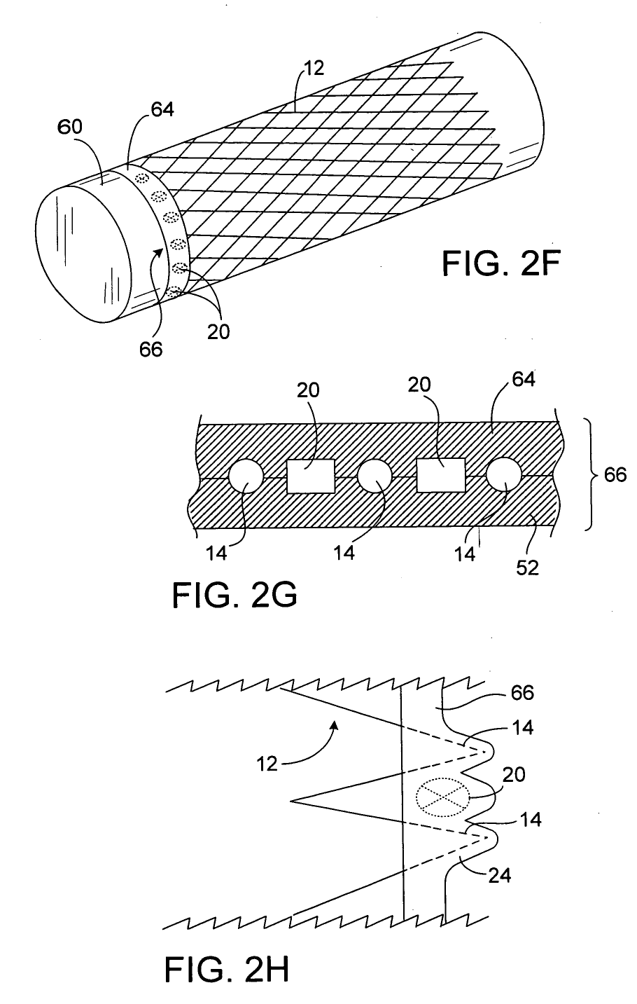

Referring to FIGS. 2A-2G, the manufacture of a stent with radiopaque markers is illustrated. Referring to FIG. 2A, radiopaque markers 20 are attached to one side 50 of a preformed polymer (e.g., expanded-polytetrafluoroethylene) strip 52. The markers 20 are adhered to polymer strip 52, for example, by spraying and/or dipping markers 20 in a low-viscosity dispersion (e.g., TFE, FEP), and then placing markers 20 on polymer strip 52. The strip 52 is heated, e.g., in an oven, such that the dispersion will cure and sinter or partially sinter with polymer strip 52. In embodiments, the temperature during heating is below the melting point of polymer strip 52. Thus, the heat can cause polymer strip 52 to soften and adhere to markers 20, without causing polymer strip 52 to melt. In embodiments, the polymer in the low-viscosity dispersion can be cross-linked and/or sintered or partially sintered to polymer strip 52, thereby securing markers 20 to polymer strip 52. For efficient manufacturing, the polymer strip to which markers 20 are attached can be longer than the circumference of the stent. The strip is then cut to a desired length to accommodate a stent of a desired size.

Referring now to FIG. 2B, the polymer strip 52 is arranged into a ring 54 (shown in FIG. 2C) after markers 20 have been adhered to polymer strip 52. While outer surface 56 of ring 54 includes markers 20, inner surface 58 of ring 54 does not include any markers 20. The diameter of the ring corresponds to the inner diameter of the stent when the stent is in a desired expanded configuration.

Referring to FIG. 2C, ring 54 is inserted onto a mandrel 60, such that inner surface 58 contacts mandrel 60. In some embodiments, mandrel 60 is a coated mandrel (e.g., coated with zirconium-nickel or titanium nitrate). In certain embodiments, a coating can help mandrel 60 to retain ring 54.

Referring now to FIGS. 2D and 2E, after ring 54 is inserted onto mandrel 60, a stent body 12 is positioned on mandrel 60, such that end 62 of stent body 12 lies on top of ring 54. Strand materials 14 are positioned between markers 20, and markers 20 are contained within voids 16. The assembly is heated to attach the ring 54 (e.g., by partial sintering) to the stent body.

Referring to FIGS. 2F and 2G, a securement layer 64 is positioned over the outer surface of the stent body and attached to ring 54. Securement layer 64 covers markers 20. Securement layer 64 can be made of, e.g., a polymer in the form of a preformed strip. The strip is formed of, e.g., the same polymer as the strip 52.

The securement layer 64 can be attached to ring 54 by adhesive-bonding (e.g., using TFE) and/or by sintering or partially sintering securement layer 64. The attachment of securement layer 64 to ring 54 forms polymer body 66, in which markers 20 are embedded. The portion of the stent body covered by the polymer body is likewise sandwiched between strip 52 and layer 64 to securely fix the markers and the polymer body 66 to the stent. (The polymer strip and the securement layer are attached to minimize gaps between the layers.)

Referring to FIG. 2H, polymer body 66 can be cut or trimmed (e.g., laser-trimmed) to reduce flaps of excess polymer material. In embodiments, polymer body 66 can be scalloped (e.g., to decrease stent end effects) and/or contoured or shaped (e.g., to smoothen polymer body 66, to enhance the biocompatibility of polymer body 66, to make polymer body 66 complement the edge of stent body 12).

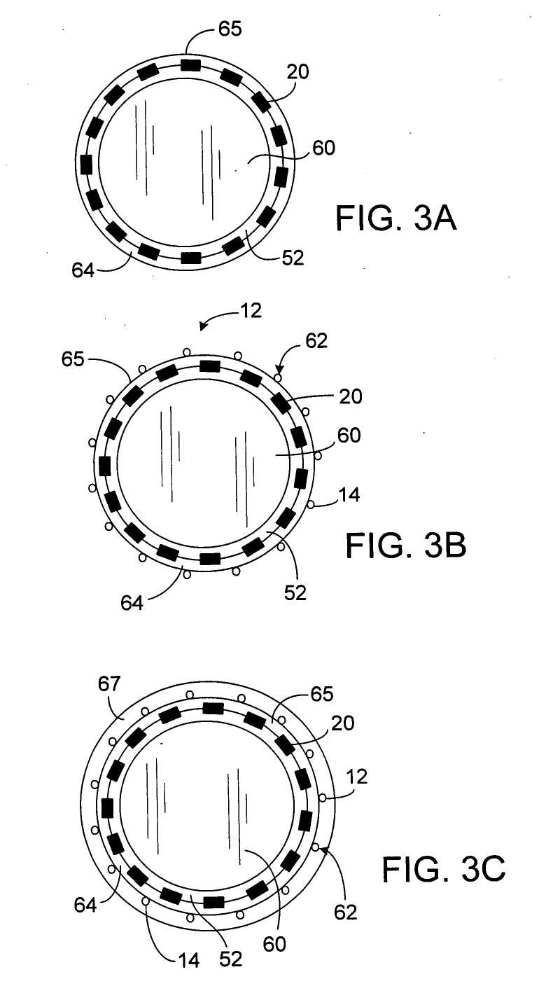

Referring now to FIGS. 3A-3C, in some embodiments a polymer ring 65 formed of markers 20 sandwiched between polymer strip 52 and securement layer 64 is inserted onto mandrel 60. Thereafter, stent body 12 is inserted onto mandrel 60, such that end 62 of stent body 12 lies on top of ring 65. Strand materials 14 of stent body 12 are positioned between the locations of markers 20 within ring 65. A second securement layer 67 is then added over ring 65 and end 62 of stent body 12, such that end 62 is sandwiched between securement layer 64 and securement layer 67.

Stent Delivery

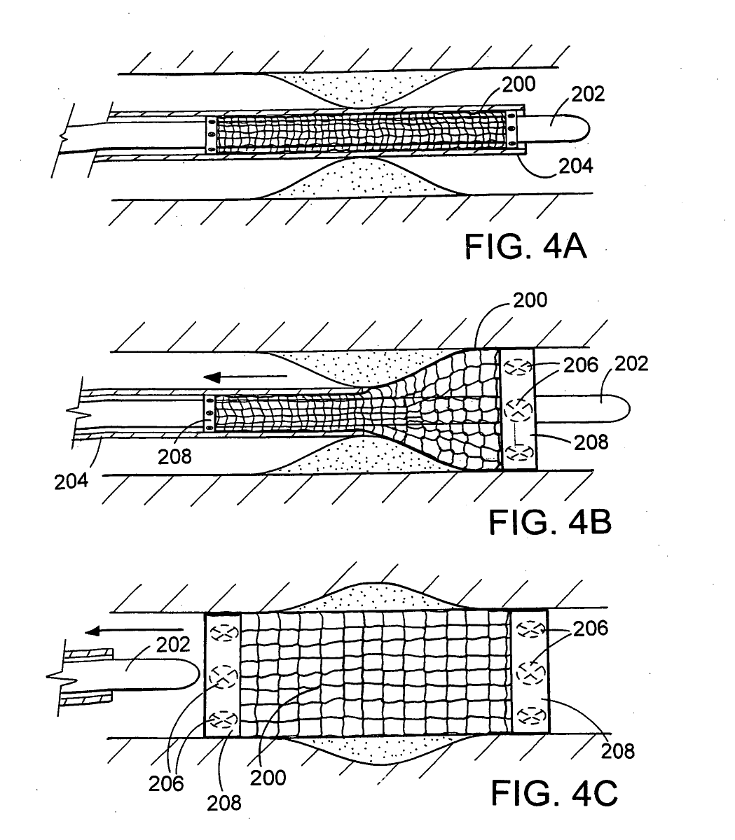

FIGS. 4A-4C show the delivery of a self-expanding stent 200. Stent 200 is deployed on a catheter 202 and covered by a sheath 204. When the target site is reached, sheath 204 is retracted and stent 200 self-expands into contact with the body lumen. Radiopaque markers 206 embedded within polymer bodies 208 at each end of stent 200 allow for determination of the location of stent 200 (e.g., by x-ray radiography).

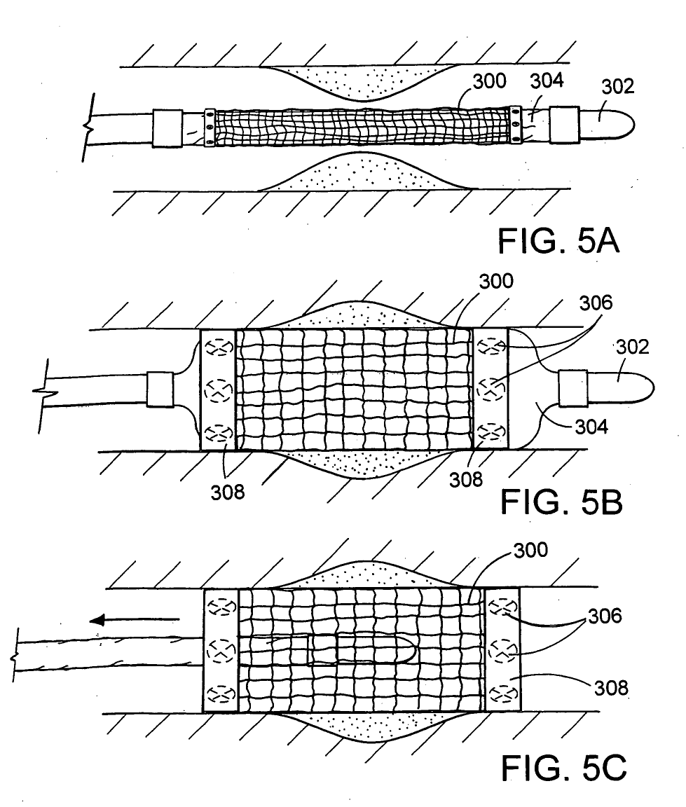

Referring now to FIGS. 5A-5C, the delivery of a balloon-expandable stent 300 is illustrated. Stent 300 is carried on a catheter 302 over a balloon 304. When the treatment site is reached, balloon 304 is expanded to expand stent 300 into contact with the lumen wall. Radiopaque markers 306 embedded within polymer bodies 308 at each end of stent 300 allow for determination of the location of stent 300.

Stent 200 and/or stent 300 can be used in vascular and/or non-vascular applications. Stent 200 and/or stent 300 can be used, for example, to treat stenoses, aneurysms, or emboli. In some embodiments, stent 200 and/or stent 300 can be used in the coronary and/or peripheral vascular system, e.g., for iliac, carotid, superior femoral artery (SFA), renal, and/or popliteal applications. In certain embodiments, stent 200 and/or stent 300 can be used in non-vascular applications. For example, stent 200 and/or stent 300 can be used in trachealtbronchial, biliary, and/or esophageal applications.

### Other Embodiments

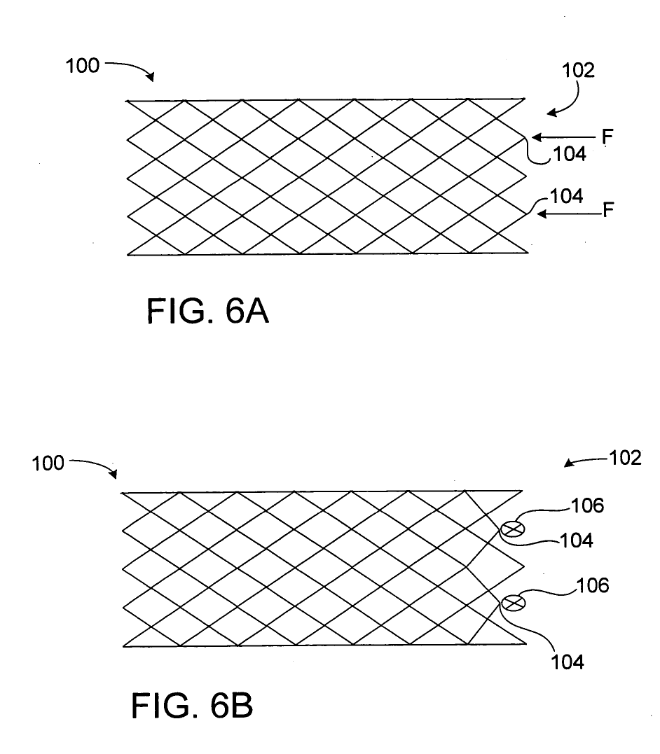

Referring to FIGS. 6A and 6B, an end 102 of the stent body of a stent 100 is modified to form a larger void volume for accommodating radiopaque markers. In FIG. 6A, forces (indicated by arrows F) are applied against points 104 to deform the stent to increase the void area to accommodate larger radiopaque markers 106 (shown in FIG. 6B). Alternatively or additionally, strand materials used to form a stent can be manipulated during the stent formation process (e.g., during weaving, knitting, crocheting) to include extra room at the edges of the stent for, e.g., radiopaque markers.

In embodiments, a stent can include a polymer body at only one of its ends, rather than at both of its ends. In certain embodiments, a stent can include a polymer body that is not located at either end of the stent. For example, a polymer body can be located at the middle of the stent body. In such embodiments, the stent can further include a polymer body at one or both of its ends, or can lack polymer bodies at either of its ends.

The polymer body can include more than one form of radiopaque material. For example, a polymer body can include embedded radiopaque markers and can have a radiopaque powder dispersed throughout it.

As a further example, a polymer body that includes radiopaque material can be incorporated into other types of medical devices. For example, the polymer body can be incorporated into various types of endoprostheses, such as a covered stent, an AAA (abdominal aortic aneurysm) stent-graft, an endograft, or a surgical vascular bypass graft, or other devices, including prosthetic venous valves and embolic protection devices and filters.

Other embodiments are within the scope of the following claims.

1. A medical stent, comprising:
a stent body comprising a generally tubular member, the generally tubular member comprising a wall that defines at least one void; and
a radiopaque material bonded to the stent body by a polymer, wherein the polymer spans the at least one void, and the radiopaque material is suspended within the at least one void.

2. The medical stent of claim 1, wherein the generally tubular member includes a pattern of voids defined through a tubular stent wall and radiopaque material is suspended within a plurality of the voids.

3. The medical stent of claim 1, wherein the radiopaque material is proximate an end of the stent body.

4. The medical stent of claim 1, wherein the polymer comprises a continuous element extending over about 50 percent or more of the circumference of the stent body.

5. The medical stent of claim 1, wherein the polymer is in the shape of a ring.

6. The medical stent of claim 5, wherein the ring has a thickness of about 125 percent of the thickness of the stent body or less.

7. The medical stent of claim 5, wherein the ring has a width of about 25 percent of the length of the stent body or less.

8. The medical stent of claim 1, wherein the polymer is a fluoropolymer.

9. The medical stent of claim 1, wherein the polymer is expanded-polytetrafluoroethylene.

10. The medical stent of claim 1, wherein the polymer encapsulates the radiopaque material.

11. The medical stent of claim 1, wherein the radiopaque material comprises a body of radiopaque metal.

12. The medical stent of claim 11, wherein the body of radiopaque metal has a thickness of about 110 percent of the thickness of the stent body or less, and about 75 percent of the thickness of the stent body or more.

13. The medical stent of claim 11, wherein the body of radiopaque metal has a thickness of from about 0.001 inch to about 0.01 inch.

14. The medical stent of claim 1, further comprising a therapeutic agent.

15. A medical stent, comprising:
a stent body defining a generally tubular member and including a pattern of voids defined through a tubular stent wall, the geometry and/or location of the voids selected to facilitate expansion and/or contraction of the stent; and
a radiopaque marker suspended within one of the voids, wherein the radiopaque marker renders the medical stent radiopaque independently of the stent body.

16. The medical stent of claim 15, wherein the radiopaque marker is located proximate an end of the stent body.

17. A method of making a stent, the method comprising:
combining a radiopaque material with a first polymer; and
attaching the first polymer to an end of a stent body defining a generally tubular member, the generally tubular member comprising a wall that defines at least one void, wherein the first polymer spans the at least one void, and the radiopaque material is suspended within the at least one void.

18. The method of claim 17, comprising providing a first strip of the first polymer, positioning a plurality of radiopaque markers on the first strip of the first polymer, and attaching the first strip to the stent body.

19. The method of claim 18, comprising positioning the radiopaque markers on the first strip at locations corresponding to voids defined by the stent body.

20. The method of claim 18, wherein attaching the first strip comprises assembling the first strip in contact with the stent body and bonding the first strip to the stent body.

21. The method of claim 20, further comprising bonding the first strip to a second strip, wherein the second strip comprises a second polymer.

22. The method of claim 21, comprising adhesive-bonding, melt-bonding, sintering or partially sintering the first strip to the second strip.

23. The method of claim 21, further comprising applying the second strip to at least one radiopaque marker to encapsulate the at least one radiopaque marker.

24. The method of claim 18, wherein the first strip is attached to the stent body by sintering or partially sintering the first strip, by melting, or by an adhesive.

25. The method of claim 17, comprising positioning at least one radiopaque marker in a void defined by the stent body.

26. The method of claim 17, wherein attaching the first polymer to an end of a stent body comprises partially sintering the first polymer to the end of the stent body.
